(19) United States
(12) Patent Application Publication
(10) Pub. No.: US 2017/0354596 A1
(43) Pub. Date: Dec. 14, 2017

(54) OIL-IN-WATER NANOEMULSIONS

(71) Applicant: Universita' Degli Studi di Pavia, Pavia (IT)

(72) Inventors: Maria Cristina Bonferoni, Pavia (IT); Giuseppina Sandri, Pavia (IT); Silvia Stefania Rossi, Pavia (IT); Franca Ferrari, Pavia (IT); Carla Marcella Caramella, Pavia (IT)

(21) Appl. No.: 15/520,155

(22) PCT Filed: Oct. 20, 2015
(86) PCT No.: PCT/IB2015/001951

§ 371 Date: Apr. 19, 2017

(30) Foreign Application Priority Data
Oct. 21, 2014 (IT) MI2014A001812

(51) Int. Cl. A61K 9/107; A61K 9/51

(52) U.S. Cl. CPC A61K 9/1075; A61K 9/5161; A61K 9/5153

(57) ABSTRACT

The present invention relates to an oil-in-water nanoemulsion comprising an oil phase comprising at least one essential oil or water-immiscible phase comprising a hydrophobic polymer and/or a hydrophobic active dispersed in an aqueous phase, said nanoemulsion comprising nanometric droplets of said essential oil or said active with an interface consisting of amphiphilic derivatives of chitosan and at least one fatty acid, obtained by ionic interaction between the deacetylated amino groups of said chitosan and the carboxylic groups of said fatty acid.

### FIELD OF THE INVENTION

The present invention relates to oil-in-water nanoemulsions and a method for the preparation thereof. In particular, the present invention relates to oil-in-water nanoemulsions comprising essential oils or water-immiscible phases comprising hydrophobic actives and/or hydrophobic polymers prepared and stabilized with the aid of amphiphilic derivatives of chitosan and fatty acids obtained by the aggregation of chitosan and at least one fatty acid by ionic interaction between the deacetylated amino groups of the chitosan and the carboxylic groups of the fatty acid.

### SUMMARY OF THE INVENTION

Essential oils are volatile liquid concentrates obtained by extraction (for example, by means of distillation or pressing) of plant material rich in aromas, such as root, leaf, flower or fruit.

Essential oils are used nowadays as fragrances, as flavourings for food and drink, as excipients in cosmetic compositions and for medicinal purposes. Hydrophobic polymers are polymeric substances, sparingly soluble in water, used as excipients in cosmetic and/or pharmaceutical compositions.

Hydrophobic actives are pharmaceutical, cosmetic or food substances sparingly soluble in water.

In a certain number of applications, particularly for topical applications to skin or mucous membranes and for some pharmaceutical preparations, in particular for the preparation of polymeric or lipidic nanoparticles, it is necessary to employ oil-in-water emulsions of essential oils or water-immiscible phases comprising hydrophobic polymers and/or hydrophobic actives.

To be used commercially, oil-in-water emulsions of essential oils or water-immiscible phases comprising hydrophobic polymers and/or hydrophobic actives must be stable over time.

To improve the stability and application or the contact with skin or mucous membranes, also to favour bioavailability and penetration, the dispersed droplets must be as small as possible, with mean diameter in the order of nanometres.

Emulsions in which the dispersed droplets have a diameter of the order of nanometres are called nanoemulsions.

Amphiphilic derivatives of chitosan and fatty acids are noted in the literature (M. C. Bonferoni et al., Ionic polymeric micelles based on chitosan and fatty acids and intended for wound healing. Comparison of linoleic and oleic acid, Eur J Pharm. Biopharm. 2014 May; 87(1):101-6) for their capacity to form polymeric micelles when dispersed in aqueous solution.

Amphiphilic derivatives of chitosan and fatty acids, consisting of an aggregation of chitosan and fatty acid by ionic interaction between the deacetylated amino groups of the chitosan and the carboxylic groups of the fatty acid, differ from the covalently modified chitosans, widely known in the prior art, wherein the amino groups of the chitosan are covalently bound to the carboxylic functions of the hydrophobic portion by formation of amide bonds.

Their use to prepare and stabilize oil-in-water emulsions of essential oils and water-immiscible phases comprising hydrophobic polymers and/or hydrophobic actives in the absence of surfactants has not been described or suggested to date.

Chitosan is a linear polysaccharide consisting of D-glucosamine and N-acetyl-D-glucosamine, linked by β(1-4) bonds. It is obtained naturally by treating chitin, generally obtained from the exoskeletons of crustacea (crabs, prawns, etc.), with an aqueous basic solution. The degree of deacetylation of the amino groups of the chitosan available commercially is between 60% and 100%. The molecular weight is generally between about 40,000 and 200,000 Dalton.

The applicant has found, surprisingly, that oil-in-water emulsions of essential oils or water-immiscible phases comprising hydrophobic polymers and/or hydrophobic actives, prepared in the presence of the aforementioned amphiphilic derivatives of chitosan and fatty acids obtained by ionic interaction, comprise droplets of essential oils and water-immiscible phases comprising hydrophobic polymers and/or hydrophobic actives, said droplets having a diameter of the order of nanometers, and being stable for several weeks.

The emulsions prepared using the aforementioned amphiphilic derivatives of chitosan and fatty acids do not require the use of further surfactants to reduce the diameter of the droplets during the preparation and/or to stabilize the emulsion during storage prior to use.

Therefore, the nanoemulsions obtained using the aforementioned amphiphilic derivatives of chitosan and fatty acids allow (i) the integrity of the cellular membrane to be respected, since they allow to avoid the use of surfactants of low molecular weight in the preparation phase, and (ii) the number of preparative phases to be reduced, since the chitosan-fatty acid interface is obtained directly in the preparation phase of the nanodroplets instead of depositing the polymer on preformed systems.

The present invention therefore relates to an oil-in-water nanoemulsion comprising an oil phase comprising at least one essential oil or at least one water-immiscible phase comprising hydrophobic polymers and/or hydrophobic actives dispersed in an aqueous phase, said nanoemulsion comprising nanometric droplets of said essential oil or said immiscible phase having an interface consisting of amphiphilic derivatives of chitosan and at least one fatty acid, wherein said amphiphilic derivatives consist of the aggregation of chitosan and at least one fatty acid by ionic interaction between the deacetylated amino groups of chitosan and the carboxylic groups of the fatty acid.

In a second aspect, the present invention relates to an oil-in-water nanoemulsion comprising an oil phase comprising at least one essential oil or at least one water-immiscible phase, said nanoemulsion comprising nanometric droplets of said essential oil or said immiscible phase having an interface consisting of amphiphilic derivatives of chitosan and at least one fatty acid, wherein said amphiphilic derivatives consist of the aggregation of chitosan and at least one fatty acid by ionic interaction between the deacetylated amino groups of chitosan and the carboxylic groups of the fatty acid.

### BRIEF DESCRIPTION OF THE FIGURES

FIG. 1 shows the values of the average diameter of the average size of the particles obtained as described in Example 2.

FIG. 2 shows the values of the average size of the particles obtained as described in Example 4.

FIG. 3 shows the values of the average diameter of the droplets of nanoemulsions 15, 17, 19 and 21 from Example 5.

FIG. 4 shows the values of the average diameter of the droplets of nanoemulsions 16, 18, 20 and 22 from Example 5.

### DETAILED DESCRIPTION OF THE INVENTION

In the present description and in the claims which follow, the expression “oil-in-water nanoemulsion” refers to an emulsion comprising nanometric droplets of essential oil or water-immiscible phase comprising a hydrophobic polymer and/or hydrophobic active dispersed in an aqueous phase.

In the present description and in the claims which follow, the expression “nanometric droplets” or “nanodroplets” refers to droplets having an average diameter of less than 1,000 nanometers. Similarly, the expression “nanometric particles” or “nanoparticles” refers to particles having an average diameter of less than 1,000 nanometers.

In the present description and in the claims which follow, the expression “amphiphilic derivatives of chitosan and at least one fatty acid” consists of the aggregation of chitosan and at least one fatty acid by ionic interaction between the deacetylated amino groups of chitosan and the carboxylic groups of the fatty acid.

In the present description and in the claims which follow, the expression “interface” represents the surface separating oil and water phases, in particular the surface of the nanometric droplets or particles.

Preferably, the oil-in-water nanoemulsion according to the present invention comprises nanometric droplets having an average diameter of less than 800 nm, more preferably less than 600 nm.

Advantageously, the oil-in-water nanoemulsion according to the present invention comprises nanometric droplets having an average diameter of less than 500 nm, more preferably less than 300 nm.

Preferably, the oil-in-water nanoemulsion according to the present invention comprises an amount of chitosan and of at least one fatty acid such that the molar ratio between the deacetylated amino groups of said chitosan and the carboxylic groups of said at least one fatty acid ranges from 1:0.1 to 1:5.

Advantageously, the oil-in-water nanoemulsion according to the present invention comprises an amount of chitosan and of at least one fatty acid such that the molar ratio between the deacetylated amino groups of said chitosan and the carboxylic groups of said at least one fatty acid ranges from 1:0.2 to 1:1.

Preferably, the oil-in-water nanoemulsion according to the present invention has a volumetric ratio between the oil phase and the aqueous phase ranging from 1:50 to 1:2, more preferably ranging from 1:25 to 1:5.

Advantageously, the oil-in-water nanoemulsion according to the present invention has a volumetric ratio between the oil phase and the aqueous phase ranging from 1:10 to 1:5.

Preferably, the oil-in-water nanoemulsion according to the present invention comprises chitosan having a deacetylation degree of the amino group higher than 60%, more preferably higher than 80%.

Advantageously, the oil-in-water nanoemulsion according to the present invention comprises chitosan having a deacetylation degree of the amino group ranging from 90% to 100%.

Preferably, the oil-in-water nanoemulsion according to the present invention comprises chitosan having a molecular weight higher than 50,000 Dalton, more preferably higher than 100,000 Dalton.

Advantageously, the oil-in-water nanoemulsion according to the present invention comprises chitosan having a molecular weight ranging from about 150,000 Dalton to 200,000 Dalton.

Preferably, the oil-in-water nanoemulsion according to the present invention comprises at least one fatty acid selected from the group comprising saturated and unsaturated fatty acids having 8 to 22 carbon atoms.

Useful examples of fatty acids useful in the present invention are caprylic acid, pelargonic acid, capric acid, lauric acid, myristic acid, palmitic acid, margaric acid, stearic acid, arachidic acid, behenic acid, palmitoleic acid, oleic acid, elaidic acid, vaccenic acid, gadoleic acid, cetoleic acid, erucic acid, linoleic acid, rumenic acid, linolenic acid, stearidonic acid, arachidonic acid, timnodonic acid, clupanodonic acid and cervonic acid.

Advantageously, the oil-in-water nanoemulsion according to the present invention comprises at least one fatty acid selected from the group comprising lauric acid, palmitic acid, stearic acid, oleic acid, gadoleic acid, erucic acid and linoleic acid.

Advantageously, the oil-in-water nanoemulsion according to the present invention comprises oleic acid or linoleic acid.

Advantageously, the oil-in-water nanoemulsion according to the present invention does not comprise low molecular weight surfactants. The expression “low molecular weight surfactant” means a surfactant having a molecular weight lower than 50,000 Dalton, preferably lower than 30,000 Dalton and more preferably lower than 10,000 Dalton.

The present invention is not particularly limited to a particular essential oil, which may be any essential oil such as, for example, Bitter Orange, Sweet Orange, Basil, Benzoin, Bergamot, Cajeput, Calendula, Chamomile, Cinnamon, Cedar, Cypress, Citronella, Coriander, Watercress, Helichrysum, Eucalyptus, Fennel, Cloves, Jasmine, Geranium, Juniper, Frankincense, Lavender Vera, Lemon, Mandarine, Peppermint, Myrrh, Myrtle, Neroli, Niaouli, Nutmeg, Oregano, Patchouli, Siberian Mountain Pine, Scotch Pine, Grapefruit, Damask Rose, Rosemary, Sage Officinalis, Clary Sage, Indian Sandalwood, Tea Tree Oil, Thyme red, Vanilla, Lemon Verbena, Ylang Ylang and Ginger.

The oil-in-water nanoemulsion according to the present invention may also comprise, as oil phase, an oil of natural origin, such as, for example, castor oil, or a natural lipophilic substance, such as, for example, alpha-tocopherol and omega-3 fatty acids.

The oil-in-water nanoemulsion according to the present invention may have, as oil phase, a water-immiscible organic solvent comprising hydrophobic polymers, preferably belonging to the category of biodegradable polymers including polyesters such as polylactides (PLA), polylactides-co-glycolides (PLGA), polycaprolactones, polyanhydrides, polyamides, polyacetals, polyketals, polycarbonates, polyiminocarbonates and polyphosphazenes, used for the preparation of biodegradable polymeric nanoparticles by a process of emulsion and evaporation or extraction of the solvent.

The oil-in-water nanoemulsion according to the present invention may have, as oil phase, a water-immiscible organic solvent comprising hydrophobic actives and mono-, di- and triglycerides used for the preparation of lipidic nanoparticles by a hot emulsion process and cooling or by evaporation of the solvent.

The present invention is not particularly limited to a particular hydrophobic active, which may be any active sparingly soluble in water such as antibiotics, particularly amphotericin B, anticancers, in particular paclitaxel, docetaxel and doxorubicin, hormones, particularly cortisone, progesterone, estradiol and testosterone, anaesthetics, particularly propofol, vitamins, in particular vitamin E and vitamin F, steroidal antiinflammatories, in particular dexamethasone and betamethasone, and so on.

The organic solvent preferably used comprises apolar organic solvents such as esters, in particular ethyl acetate, ethers, particularly diethyl ether, halogenated hydrocarbons, particularly chloroform and methylene chloride, aliphatic hydrocarbons, particularly hexane and cyclohexane, and aromatic hydrocarbons, particularly benzene, toluene and xylene.

The oil-in-water nanoemulsion according to the present invention may be useful in the production of liquid formulations or medical devices (mouthwashes, vaginal douches) or semi-solids (gels, creams, unguents, pastes) to be used for topical application, by the cutaneous route or to apply to mucous membranes (oral, nasal, rectal, vaginal, conjunctival).

The examples which follow are intended to further illustrate the present invention without being limiting in any way.

### EXAMPLES

### Example 1

### Preparation of Chitosan Hydrochloride

With constant stirring on a magnetic plate, 2 grams of chitosan from Sigma-Aldrich (MW=187,578 g/mol) are added gradually to 100 ml of a solution of 1M HCl (Carlo Erba, Milano, Italy) in double distilled H₂O. On complete dissolution of the chitosan, the solution was transferred to a dialysis membrane, previously rehydrated by means of boiling in deionized H₂O for a period of 15 minutes.

The system was maintained under stirring on the plate in an appropriate volume of double distilled H₂O, replaced every 30 minutes for the first 3 hours and then maintained for about 12 hours. Aliquots of the solution, after freezing, were lyophilized (Heto Dry Winner, Analitica de Mori, Milano, Italy) thus obtaining the HCl salt of chitosan (chitosan hydrochloride).

### Example 2

### Preparation of Nanoparticles of Poly(Lactide-Co-Glycolide) (PLGA) by Ethyl Acetate Nanoemulsions

Various oil-in-water emulsions were prepared using 3 ml of aqueous phase comprising 1 mg/ml chitosan hydrochloride and variable amounts of oil phase composed of ethyl acetate comprising 24 mg/ml PLGA (250 μl or 500 μl) and oleic acid hydrophobic agent in a (1:1) stoichiometric ratio with the amino groups of the chitosan or a ratio five-fold lower (1:0.2). The emulsions were maintained under stirring using an Ultraturrax for 10 minutes at two different speeds (9,500 or 13,500 revolutions per minute). The ethyl acetate was removed from the nanoemulsions at 40° C. with magnetic stirring to obtain polymeric nanoparticles.

Six samples of polymeric nanoparticles 1-6 were obtained using the values for each variable reported in Table 1 below.

TABLE 1

| | Ratio chitosan/ | Amount of oil | Stirring speed |
|---|---|---|---|
| Emulsion | hydrophobic agent | phase (μl) | (revolutions/minute) |
| 1 | 1:0.2 | 250 | 9,500 |
| 2 | 1:0.2 | 500 | 9,500 |
| 3 | 1:1 | 250 | 9,500 |
| 4 | 1:1 | 500 | 9,500 |
| 5 | 1:1 | 250 | 13,500 |
| 6 | 1:1 | 500 | 13,500 |

The nanoparticles were centrifuged for 10 minutes at 3,000 revolutions/minute and then size analysis was performed on the supernatant by photon correlation spectroscopy (PCS) using the instrument N5 Submicron Particle Size Analyzer from Beckman Coulter using scattering angles of 30° and 90°.

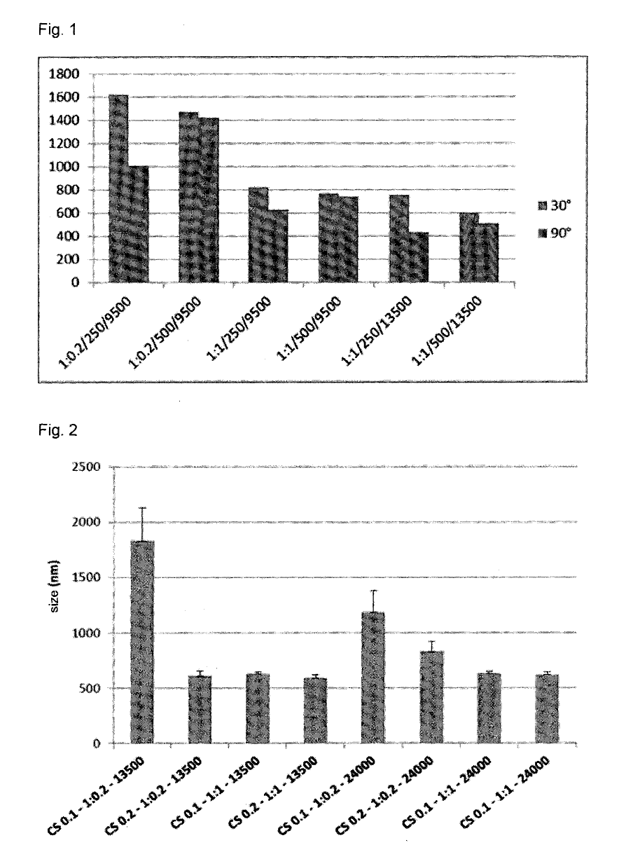

The values for the average diameter of the particles thus obtained are illustrated in FIG. 1.

The results obtained have shown that the particle diameter is mainly influenced by the ratio between chitosan and hydrophobic agent, whereas the other variables have a relatively low influence. In particular, it is observed that all the particles obtained having a (1:1) stoichiometric ratio between chitosan and hydrophobic agent showed an average diameter less than one micrometer, and between 400 and 800 nm.

### Example 3

### Preparation of Amphiphilic Derivatives of Chitosan and Oleic Acid

To 3 ml of aqueous phase comprising chitosan hydrochloride at various concentrations (0.1 and 0.2 mg/ml) was added dropwise oleic acid, dissolved in acetone at a concentration of 10 mg/ml, at a (1:1) stoichiometric ratio with the amino groups of the chitosan or a ratio five-fold lower (1:0.2). The acetone was evaporated at room temperature leaving a dispersion of chitosan and oleic acid in water.

### Example 4

### Preparation of Nanoparticles of Poly(Lactide-Co-Glycolide) (PLGA) by Ethyl Acetate Emulsions

3 ml of double distilled water and 2.6 ml of ethyl acetate comprising 4.6 mg/ml PLGA were mixed using an Ultraturrax at 9,500 revolutions/minute, and a volume equal to 16 ml of the aqueous dispersion of chitosan and oleic acid prepared as described in example 3 was added. The emulsion was maintained under stirring using an Ultraturrax for 10 minutes at two different speeds (13,500 or 24,000 revolutions per minute). The ethyl acetate was gradually removed at about 40° C. under magnetic stirring.

Eight samples of polymeric particles 7-14 were obtained using the values for each variable reported in Table 2 below.

TABLE 2

| | Chitosan | Ratio | |
|---|---|---|---|
| | concentration | chitosan/hydrophobic | Stirring speed |
| Emulsion | (mg/ml) | agent | (revolutions/minute) |
| 7 | 0.1 | 1:0.2 | 13,500 |
| 8 | 0.2 | 1:0.2 | 13,500 |
| 9 | 0.1 | 1:1 | 13,500 |
| 10 | 0.2 | 1:1 | 13,500 |
| 11 | 0.1 | 1:0.2 | 24,000 |
| 12 | 0.2 | 1:0.2 | 24,000 |
| 13 | 0.1 | 1:1 | 24,000 |
| 14 | 0.2 | 1:1 | 24,000 |

The values for the average diameter of the particles thus obtained were determined by photon correlation spectroscopy (PCS) using the instrument N5 Submicron Particle Size Analyzer from Beckman Coulter using scattering angles of 30° and 90° and are illustrated in FIG. 2.

The results obtained confirmed that the stirring speed does not particularly influence the diameter of the particles, but the diameter is particularly influenced by the concentration of chitosan and the ratio between chitosan and hydrophobic agent. In fact, it was observed that only emulsions 13 and 17, with the lowest concentrations of chitosan and the lowest ratio between chitosan and hydrophobic agent, showed an average diameter above one micrometer, whereas all the other polymeric nanoparticles showed an average diameter less than one micrometer, and between 600 and 800 nm.

### Example 5

### Preparation of Emulsions of Citronella Essential Oils

Four different aqueous solutions of chitosan hydrochloride at various concentrations (0.5-1-2-5-mg/ml) and three different solutions of oleic acid in acetone (10-20-50 mg/ml) were prepared.

To 3 ml of chitosan hydrochloride solution was added a solution of oleic acid in acetone calculated to give a stoichiometric molar ratio of 1:1 between the deacetylated amino groups of the chitosan and the carboxylic groups of the oleic acid.

Immediately before the addition to the aqueous solution of chitosan hydrochloride, the volume of solution of oleic acid was admixed with a solution of citronella essential oil in acetone containing an amount of essential oil equal to 50% or to 100% of the weight of chitosan hydrochloride and oleic acid.

The solution in acetone was added dropwise under magnetic stirring. At the end of the addition, the acetone was evaporated under a flow of nitrogen at room temperature for about 40 minutes and the volume was restored with double distilled water.

The emulsions were subjected to centrifugation for 15 minutes at 3,000 revolutions/minute and then to sonication for 15 minutes at room temperature using an Elmasonic S80H device, Elma Hans Schmidbauer GmbH & Co.

Eight nanoemulsions 15-22 were obtained, using for each variable the values reported in Table 3 below.

TABLE 3

| | Chitosan | Essential oil |
|---|---|---|
| Suspension | concentration (mg/ml) | concentration (%) |
| 15 | 0.5 | 50 |
| 16 | 0.5 | 100 |
| 17 | 1.0 | 50 |
| 18 | 1.0 | 100 |
| 19 | 2.0 | 50 |
| 20 | 2.0 | 100 |
| 21 | 5.0 | 50 |
| 22 | 5.0 | 100 |

The nanoemulsions were then subjected to size analysis by photon correlation spectroscopy (PCS) using the instrument N5 Submicron Particle Size Analyzer from Beckman Coulter using scattering angles of 30° and 90°. The size analysis was performed on freshly prepared nanoemulsions and on nanoemulsions stored at room temperature for defined periods of up to three months.

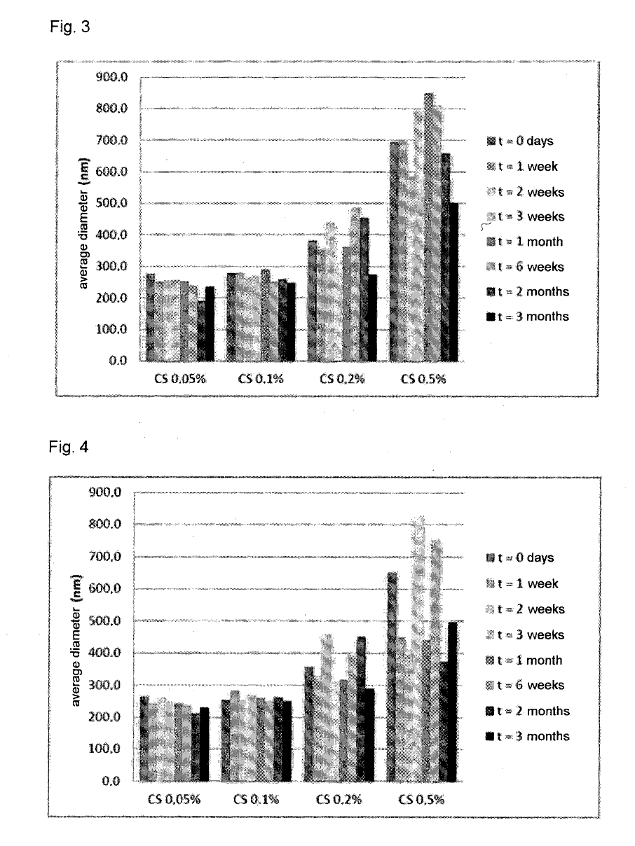

The values of the average diameter of the suspensions measured at the intervals indicated are illustrated in FIGS. 3 and 4.

Nanoemulsions 15-18 showed an average diameter of less than 300 nm.

Nanoemulsions 19 and 20 showed a slightly higher average diameter but still less than 500 nm over the whole three month storage.

Since from time zero, nanoemulsions 21 and 22 showed a higher average diameter than the other suspensions, but remained stable during the three month storage.

1. An oil-in-water nanoemulsion comprising an oil phase comprising at least one essential oil or at least one water-immiscible phase comprising a hydrophobic polymer and/or a hydrophobic active dispersed in an aqueous phase, said nanoemulsion comprising nanometric droplets of said essential oil or said water-immiscible phase, wherein said droplets have an interface consisting of amphiphilic derivatives of chitosan and at least one fatty acid, wherein said amphiphilic derivatives consist of aggregation of said chitosan and said at least one fatty acid by ionic interaction between the deacetylated amino groups of said chitosan and the carboxylic groups of said fatty acid.

2. The oil-in-water nanoemulsion according to claim 1, wherein said nanometric droplets have an average diameter lower than 1,000 nm.

3-4. (canceled)

5. The oil-in-water nanoemulsion according to claim 1, wherein the molar ratio between the deacetylated amino groups of said chitosan and the carboxylic groups of said at least one fatty acid ranges from 1:0.1 to 1:5.

6. The oil-in-water nanoemulsion according to claim 1, wherein the molar ratio between the deacetylated amino groups of said chitosan and the carboxylic groups of said at least one fatty acid ranges from 1:0.2 to 1:1.

7. The oil-in-water nanoemulsion according to claim 1, wherein said oil-in-water nanoemulsion has a volumetric ratio between the oil phase and the aqueous phase ranging from 1:50 to 1:2.

8. The oil-in-water nanoemulsion according to claim 1, wherein said chitosan has a deacetylation degree of the amino group higher than 60%.

9. The oil-in-water nanoemulsion according to claim 1, wherein said chitosan has a molecular weight higher than 50,000 Dalton.

10. The oil-in-water nanoemulsion according to claim 1, wherein said oil-in-water nanoemulsion does not comprise surfactants having a molecular weight lower than 50,000 Dalton.

11. The oil-in-water nanoemulsion according to claim 1, wherein said oil phase consists of an essential oil.

12. The oil-in-water nanoemulsion according to claim 1, wherein said oil phase consists of a water-immiscible phase comprising a hydrophobic polymer.

13. Nanoemulsion according to claim 12, wherein said hydrophobic polymer is a biodegradable polymer selected from the group consisting of polylactides (PLA), polylactides-co-glycolides (PLGA), polycaprolactones, polyanhydrides, polyamides, polyacetals, polyketals, polycarbonates, polyiminocarbonates and polyphosphazenes.

14. The oil-in-water nanoemulsion according to claim 1, wherein said oil phase consists of a water-immiscible phase comprising a hydrophobic active.

15. Nanoemulsion according to claim 14, wherein said hydrophobic active is selected from the group consisting of antibiotics, anticancers, hormones, anaesthetics, vitamins and steroidal anti-inflammatory drugs.

16. A method for the preparation of an oil-in-water nanoemulsion comprising an oil phase comprising at least one essential oil or at least one water-immiscible phase comprising a hydrophobic polymer and/or a hydrophobic active dispersed in an aqueous phase, said method comprising a step of forming nanometric droplets of said essential oil or said water-immiscible phase in the presence of amphiphilic derivatives of chitosan and at least one fatty acid obtained by ionic interaction between the deacetylated amino groups of said chitosan and the carboxylic groups of said fatty acid.

17. Method according to claim 16, wherein the molar ratio between the deacetylated amino groups of said chitosan and the carboxylic groups of said at least one fatty acid ranges from 1:0.1 to 1:5.

18. Method according to claim 16, wherein the molar ratio between the deacetylated amino groups of said chitosan and the carboxylic groups of said at least one fatty acid ranges from 1:0.2 to 1:1.

19-20. (canceled)

21. An oil-in-water nanoemulsion comprising an oil phase dispersed in an aqueous phase, said oil phase comprising at least one essential oil or at least one water-immiscible phase, said nanoemulsion comprising nanometric droplets of said essential oil or said water-immiscible phase, wherein said nanometric droplets have an interface consisting of amphiphilic derivatives of chitosan and at least one fatty acid, wherein said amphiphilic derivatives consist of aggregation of said chitosan and said at least one fatty acid by ionic interaction between the deacetylated amino groups of said chitosan and the carboxylic groups of said fatty acid.

22. The nanoemulsion according to claim 21, wherein said essential oil is selected from the group consisting of essential oil of Bitter Orange, Sweet Orange, Basil, Benzoin, Bergamot, Cajeput, Calendula, Chamomile, Cinnamon, Cedar, Cypress, Citronella, Coriander, Watercress, Helichrysum, Eucalyptus, Fennel, Cloves, Jasmine, Geranium, Juniper, Frankincense, Lavender Vera, Lemon, Mandarine, Peppermint, Myrrh, Myrtle, Neroli, Niaouli, Nutmeg, Oregano, Patchouli, Siberian Mountain Pine, Scotch Pine, Grapefruit, Damask Rose, Rosemary, Sage Officinalis, Clary Sage, Indian Sandalwood, Tea Tree Oil, Thyme red, Vanilla, Lemon Verbena, Ylang Ylang and Ginger.

23. Nanoemulsion according to claim 21, wherein said water-immiscible phase is an organic solvent selected from the group consisting of esters, ethers, halogenated hydrocarbons, aliphatic hydrocarbons and aromatic hydrocarbons.

24. Nanoemulsion according to claim 21, wherein said water-immiscible phase is an oil or a natural lipophilic substance.
